(19) United States
(12) Patent Application Publication
(10) Pub. No.: US 2020/0193496 A1
(43) Pub. Date: Jun. 18, 2020

(54) CONSUMER ACCESS TO PERSONAL, HEALTH-RELATED DATA IN REAL-TIME TO SUPPORT COMPREHENSION, SAFE SELF-SELECTION, AND APPROPRIATE USE OF HEALTH CARE PRODUCTS AS PER AN FDA-APPROVED LABEL METHOD AND SYSTEM

(71) Applicant: Biograph Inc., Florham Park, NJ (US)

(72) Inventors: Mary Alice LAWLESS, Morristown, NJ (US); Frederick T. LAWLESS, III, Morristown, NJ (US); David Cutler BRADFORD, Salt Lake City, UT (US); John J. GOODWIN, Flemington, NJ (US); David McCAMMON, Nordland, WA (US); Joseph McGOVERN, Long Valley, NJ (US); Brent Cutler PAGE, Boutiful, UT (US); Alpesh PATEL, Totowa, NJ (US); Clark Andrew RICHARDSON, Centerville, UT (US); Dennis TZE, Ridgewood, NJ (US)

(21) Appl. No.: 16/798,743

(22) Filed: Feb. 24, 2020

Related U.S. Application Data
(63) Continuation-in-part of application No. 15/841,729, filed on Dec. 14, 2017.
(60) Provisional application No. 62/433,884, filed on Dec. 14, 2016.

(51) Int. Cl. G06Q 30/06; G06Q 30/00; G16H 80/00; G16H 70/40; G16H 10/60; G16H 10/40; G16H 70/20; G16H 20/10; G06Q 20/40; G16H 10/20; G09B 19/00

(52) U.S. Cl. CPC G06Q 30/0607; G06Q 30/0623; G06Q 30/018; G06Q 30/0641; G06Q 30/016; G16H 80/00; G09B 19/00; G16H 10/60; G16H 10/40; G16H 70/20; G16H 20/10; G06Q 20/405; G16H 10/20; G16H 70/40

(57) ABSTRACT

A consumer health care product self-selection system, including: a processor coupled to a memory containing program code; a network interface configured to provide access to: a user interface configured to receive input from and providing output to a consumer; one or more databases; a product distribution system; and a product support system; the processor is configured to: provide information of one or more products to the consumer; receive selection of the product; determine the consumer's eligibility of purchasing the selected product based on at least one of: consumer data and product data retrieved from the one or more databases, and data entered by the consumer; when eligible, direct the consumer to the product distribution system to purchase the selected product; and when ineligible, direct the consumer to the product support system to provide product support information of the selected product to the consumer and access to a healthcare professional.

### CROSS REFERENCE TO RELATED APPLICATIONS

This application is a continuation-in-part under 35 U.S.C. § 120 of U.S. application Ser. No. 15/841,729, filed Dec. 14, 2017, which claims the benefit of U.S. Provisional Application No. 62/433,884 filed on Dec. 14, 2016, the contents of each of which are herein incorporated by reference.

### FIELD OF THE INVENTION

One embodiment of this invention relates to a method and system to deliver support for consumer label comprehension, safe self-selection and appropriate use of Consumer Health Care (CHC) products and the data-driven distribution process using technology.

### BACKGROUND

Consumer Health Care (CHC) products include over-the-counter (OTC) medicines, devices and drug-device combination products which are approved by the FDA for consumer access. CHC products can be directly purchased by consumers and used without a prescription. These products are deemed safe and effective when it is proven that they can be selected and used by consumers in accordance with an FDA-approved Drug Facts Label (DFL) or other labeling regulated by FDA.

CHC products play an increasingly vital role in America's health care system as the nation struggles to achieve public health goals for disease prevention and chronic condition management. OTCs provide access to care at the lowest total cost to the consumer and with the least burden to the U.S. health care system. The candidates for a next generation of CHC products are especially exciting in terms of their anticipated contribution to public health. OTC access to products for cardiovascular risk management or respiratory disease prevention, for example, would enable consumers to take significant new action in the management of their health. But, to approve OTC access for these products, FDA will require methods and systems to educate and enable consumer safe self-selection and correct use, such that the purpose of the FDA-approved product label and any extensions of the label (labeling) are fully served, which is to make it easier for consumers to read and understand product information as it applies to them, and to use the products safely and effectively.

According to FDA, approval for next generation OTCs is possible with a consumer-driven safe self-selection method that enables self-screening, supported by a guidelines-based algorithm and personal clinical data, which functions as a learning system. One embodiment of the present invention accomplishes this goal by vertically integrating the OTC access process with U.S. health care data systems and connecting the consumer to their personal healthcare data in a dynamic experience of the specific FDA-approved labeling for the product. The required data is available in the repositories of various fiduciaries (data providers) across the health care network. One embodiment of the present invention will integrate with these repositories to access individual consumer health data and to deliver such data to support consumer safe selection and use in compliance with approved labeling.

The Federal Food, Drug, and Cosmetic Act requires that a product be shown to be safe for use without the professional supervision of a practitioner licensed by law to administer the drug product and that its labeling “is likely to be read and understood by the ordinary individual under customary conditions of purchase and use” (21 U.S.C. 352(c)). In the development process of new OTC medications, FDA may require consumer studies as a complement to retrospective safety and efficacy data. The current consumer studies in an OTC regulatory pathway may include Label Comprehension Study (LC), Self-Selection Study (SS), Actual Use Trial (AUT), and Human Factor Study (HF). An LC assesses the extent to which the consumer understands the labeling information on a nonprescription drug product and how the consumer then applies this information when making drug product selection and usage decisions. An SS determines and provides evidence of consumers' ability to select an OTC product for the appropriate purpose and follow the labeling such that it is used appropriately. An AUT is a clinical study wherein participants receive and use the study drug to simulate its OTC use and predict whether it will be used properly, safely and effectively in the OTC setting. An HF is conducted with any combination drug product or device in order to assess the adequacy of the user interface to eliminate or mitigate potential use-related hazards.

In the Federal Register of Mar. 17, 1999 (64 FR 13254), the FDA published a final regulation (§ 201.66) establishing standardized content and format for the labeling of OTC products (Drug Facts labeling). By law, OTC medicine labeling must include all the information that an ordinary consumer needs for the safe and effective use of the product; and “labeling” means all labels, and other written, printed, or graphic matter (1.) upon any article or any of its containers, or (2.) accompanying such article. The FDA issued guidance in 2008 which is intended to facilitate compliance with the labeling requirements in § 201.66. which requires that all OTC drug product labeling contain the following information about the drug product, organized according to the following headings and presented in the following order:

1. Title (Drug Facts or Drug Facts (continued))

2. Active ingredient(s)

3. Purpose(s)

4. Use(s)

5. Warning(s)

6. Directions

7. Other information

8. Inactive ingredients

9. Questions? or Questions or comments? (optional)

The present invention, in one embodiment, with FDA requirements for approval and labeling, will use consumer health data and interact with the consumer to enable safe self-selection and correct use of the CHC product.

### SUMMARY

One embodiment of this invention relates to a method and system to deliver support for label comprehension, safe self-selection and appropriate use of consumer health care products and the data-driven distribution process using technology and recording of new data generated by this process into data repositories. The method and system provide safe self-selection, appropriate use and distribution of CHC products by utilizing consumer personal health data as it relates to a product-specific label, delivered through an integrated system which functions to inform the safe selection and use process.

In the current OTC product distribution environment, consumers must rely on their own judgment to self-select the product, and there is no safeguard for improper choice or misuse of the product, once selected. Furthermore, there is no feedback or educational information provided to the consumers regarding their product selection. Also, there is no obvious method for consumers to ask questions they may have about OTC products before selection and use. Vertically integrating the OTC access process with the repositories of various data providers across the U.S. health care network, and delivering the FDA-approved labeling information in a dynamic experience allows for a unique process of the self-selection, distribution and education functions in one OTC access system. Thus an embodiment of the present invention significantly improves the system and process of providing OTC health care products to consumers in a safe and protected manner. It properly aligns the consumer's profile with the FDA-approved labeling of the product. This delivers safe self-selection and distribution of OTC products in the health care industry.

One embodiment of the present invention provides a consumer health care product self-selection system, including: a processor coupled to a memory containing program code; and a network interface configured to provide access to: a user interface configured to receive input from and providing output to a consumer; one or more databases; a product distribution system; and a product support system; wherein the processor is configured to: provide information of one or more products to the consumer via the user interface; receive selection of the product by the consumer; determine the consumer's eligibility of purchasing the selected product based on at least one of: consumer health data and product data retrieved from the one or more databases, and data entered by the consumer via the user interface; when the consumer is determined to be eligible, direct the consumer to the product distribution system to purchase the selected product; and when the consumer is determined to be ineligible, direct the consumer to the product support system to provide product support information of the selected product to the consumer, as well as access to a healthcare professional, if necessary or desired.

One embodiment of the present invention provides a method of providing access of a consumer health care product for safe self-selection and distribution, including: providing information of one or more products to a consumer via a user interface; receiving selection of a product by the consumer; determining the consumer's eligibility of purchasing the selected product based on at least one of: consumer data and product data retrieved from one or more databases, and data entered by the consumer via the user interface; when the consumer is determined to be eligible, directing the consumer to a product distribution system to purchase the selected product; and when the consumer is determined to be ineligible, directing the consumer to a product support system to provide product support information of the selected product to the consumer, as well as access to a healthcare professional, if necessary or desired.

As discussed above, FDA determines the study program for each OTC application on a case by case basis, and there are four typical studies which may be required by FDA as part of the approval process for OTCs (typically over a period of 5+ years): (1) Label Comprehension Study; (2) Safe Self-Selection; (3) Actual Use Trial; and (4) Human Factors Study.

Disclosed embodiments of method and system of providing access of a consumer health care product in this application may be implemented to support one or more of the typical studies (1)-(3) or other studies that may be required by FDA as part of the approval process. All FDA approvals for OTC access are linked to the product-specific Drug Facts Label (DFL). Since the embodiments utilizes consumer personal health data as it relates to a product-specific label to perform qualification checks and provide product support and education to the consumers, the success rates in these studies are likely to increase, as the data-driven process helps reduce or eliminate many human errors.

### BRIEF DESCRIPTION OF THE DRAWINGS

FIG. 1 is one embodiment of a method and system for label comprehension, safe self-selection, appropriate use, and distribution of CHC products;

FIG. 2 is one embodiment of a method implemented on CHC product education and support to consumers of FIG. 1;

FIG. 3 is one embodiment of a process implemented on CHC eligibility as part of the method of FIG. 1; and

FIG. 4 is one embodiment of a process implemented on CHC distribution and learning system as part of the method of FIG. 1.

### DETAILED DESCRIPTION OF THE PREFERRED EMBODIMENTS

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

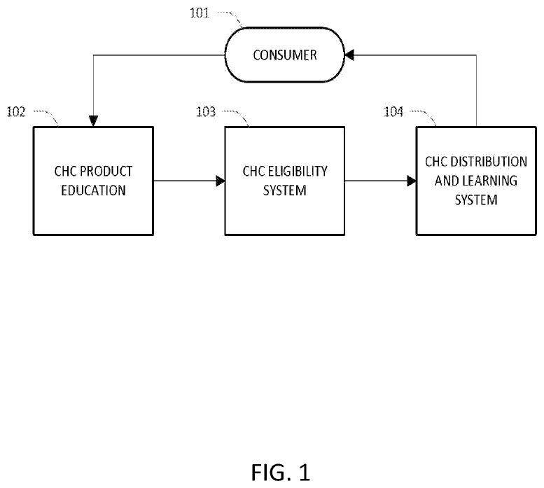

FIG. 1 is one embodiment of the present invention relates to the method and system to create support for consumer safe self-selection, label comprehension, and appropriate use of CHC products. A consumer 101 will be informed of a CHC product. Information about the CHC product will come from sources including but not limited to online, in-store, traditional media and print 102. The consumer is stimulated by the information about the CHC product to take action toward purchase. In one embodiment, the CHC product is selected on the CHC eligibility system 103. The system includes a processor coupled with a memory containing program code, and a network interface. The interface has access, but not limited to consumer interfaces, health care databases, product distribution systems, product support systems. The system 103 uses criteria based on the CHC product DFL, other product purchase and distribution rules, consumer health data, and questions to process the outcome to determine eligibility for purchase and will integrate that outcome with a distribution partner 104 to consummate the sale and distribution of the CHC product. A learning system of education and support documents 104 are provided to the consumer to further guide, inform and support adherence on the CHC product.

A set of purchase and distribution rules will be available to prevent unsafe use or gaming of the system—e.g. limits on purchase quantities, allowed purchase frequency, tracking of multiple attempts to qualify, same shipping address from multiple accounts, etc. In one embodiment, during the eligibility check, the consumer health data retrieved will be also checked against one or more of the purchase rules.

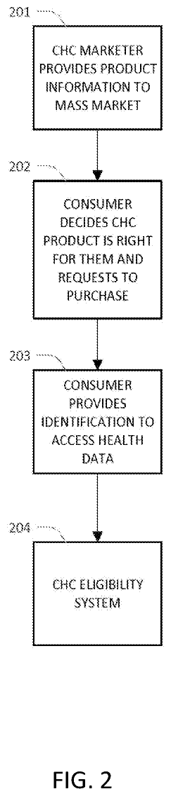

FIG. 2 illustrates one embodiment of a block diagram of a method on the CHC product education and support to consumers. In the embodiment, the CHC product marketer provides product information in many media formats 201. The CHC product information presented to the consumers explains what use the CHC product is for, who qualifies for use and how to purchase the CHC product. The safe self-selection process begins when a consumer receives information about the CHC product 201. The consumer decides if the CHC product is right for them and is directed to the safe self-selection system 202. Access to the safe self-selection system may include, but is not limited to, an electronic device that is connected to the internet or phone. In one embodiment, the consumer (not shown) will provide information to identify and access their health data 203. This information is provided into the CHC eligibility system 204.

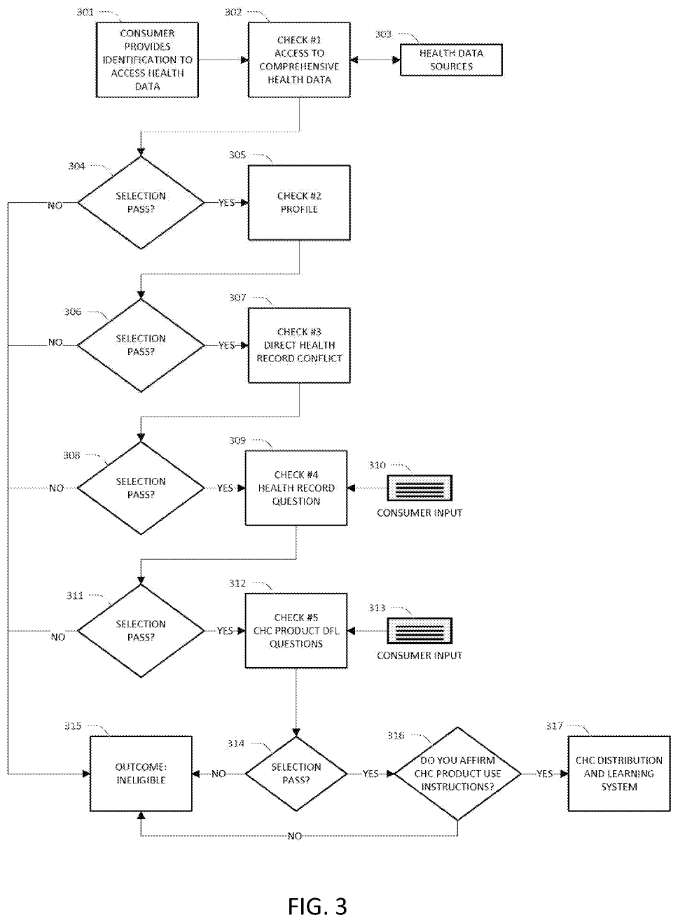

FIG. 3 is one embodiment of a block diagram of one embodiment of the CHC eligibility system 103 in FIG. 1. Each CHC product will have unique eligibility criteria based on the approved CHC product label and a set of purchase and distribution rules. The label will be used to determine the set of one or more requirement predicates. The requirement predicate is a logical expression that computes a value of “true”, meaning the predicate passes, or “false”, meaning the predicate fails. The consumer provides the necessary identification information 301 for the system to retrieve the consumer's health record from the health record providers 303. The consumer will input into the system their identification. This identification could include but is not limited to First Name, Last Name, Date of Birth, Gender, Street Address, Zip Code, Rx BIN, Group number, and Health ID number. Health data sources 303 provide the input data requirements about the consumer used to evaluate the requirement predicates. Health data sources 303 could include but are not limited to, Pharmacy Benefit Managers (PBMs), health plans, pharmacy data providers, Electronic Health Records (EHR), lab test companies, and biometric data sources and other health records. In the embodiment of FIG. 3, based on the approved CHC product label the system will do five checks, for example. It is contemplated that more or fewer checks and different checks or different sets of checks may be used. Each check will have a set of predicates to ensure proper safe self-selection. If selection pass is deemed a “no”, the outcome is ineligible 315 for the CHC product purchase. The consumer has the option to connect directly to a healthcare professional via but not limited to chat, video conference, telehealth connection or telephone. If selection pass is deemed a “yes”, the outcome continues to the next check. The selection pass will indicate a need for educational material provided to the consumer based on the specific check at which the consumer failed. In the embodiment, Check #1 Access to Comprehensive Health Data 302, will assess the availability of a comprehensive set of health data on the consumer from the health record sources 303. The criteria will ensure that the minimum necessary information on the consumer health data is collected and matches with the consumer-provided identification. This will include but is not limited to First Name, Last Name, Date of Birth, Gender, Street Address and Zip Code. Other checks may include reference against health data sources that do not share information. If selection pass 304 is deemed a “no”, the outcome is ineligible 315. If the selection pass 304 is deemed a “yes”, the outcome continues to Check #2 Profile 305.

Check #2 Profile 305 determines if the consumer profile predicate matches the profile of the CHC product DFL. If selection pass 306 is deemed a “no”, the outcome is ineligible 315. The consumer will be provided information on their ineligibility. For example, if the DFL of a CHC product label states the product is only to be used by a male between the age of 45 and 74, the Check #2 will check the consumer's data. If the consumer's age is 39, the consumer is ineligible. The system will notify the consumer they do not meet the criteria and supply information and education the consumer can use to talk to a doctor for further support. If the selection pass 306 is a deemed a “yes”, the outcome is eligible and continues on to Check #3 Direct Health Record Conflict 307.

Check #3 Direct Health Record Conflict predicate 307 is designed to match the consumer's health data with the list of drugs and conditions in the Warnings section of the CHC product's DFL. If the system finds a conflict in the consumer's data history to the list CHC product DFL, then the selection pass 308 is deemed a “no”. If no conflict is found then the selection pass 308 is a “yes”. For example, if the DFL warnings of a CHC product state the consumer cannot take the product if they also take nitroglycerine, nitroglycerine will be on the master list of contraindicated drugs. If nitroglycerine is present in the consumer's prescription health record, the consumer will not be eligible to purchase the CHC product. Education around the contraindication is provided, and the consumer is advised to go see a doctor for support.

Check #4 Health Record History Question predicate 309 is designed to ask the consumer clarifying questions about prescriptions that are listed in their Rx history that are used to treat a certain condition. The condition is based on the CHC product label and brings the attention to the consumer's answer. For example, a consumer's health data shows the consumer has been prescribed acetylsalicylic acid. Acetylsalicylic acid may be prescribed for people with certain diseases, including hypertension. The CHC product's DFL states people with hypertension should not use CHC product. People with an acetylsalicylic acid prescription in their record may also have hypertension, so the question will be prompted by the system, “Are you using acetylsalicylic acid to treat hypertension?” If the consumer responds “yes” the selection pass 311 is a “no”, and the consumer would get information to talk to their doctor. Simultaneously, the consumer will be given the option of connecting directly to a healthcare professional via but not limited to chat, video conference, telehealth connection or telephone. If the consumer answers “no”, the selection pass 311 is a “yes”, and they continue to the next check.

Check #5 CHC Product DFL Questions predicate 312 is designed to ask the consumer questions that are not reflected in their health data. These questions are based on the CHC product DFL that every consumer must answer and are mandatory regardless of the consumer's health data. If, based on the answers the consumer provides, the eligible pass 314 is a “no”, then information and education will be provided to the consumer. Throughout, the consumer will be given the option of connecting directly to a healthcare professional via but not limited to chat, video conference, telehealth connection or telephone. If the eligible pass 314 is a “yes”, then they will affirm understanding of the DFL and instructions for use 316. For example, the CHC product DFL states to see a doctor before taking the drug if you get dizzy when standing up. The system would prompt the question, “Do you get dizzy when standing up?” If the consumer responds “yes”, the selection pass 314 is a “no”, and the consumer will get information to talk to their doctor. If the consumer answers “no”, the selection pass 314 is a “yes”, and the consumer will continue onto the next section 316.

Upon selection pass 314 is a “yes”, the system will ask the consumer to affirm they understand the DFL and instructions for use of the CHC product 316. If the response is a “no”, the consumer is informed about their ineligibility 315. If response is “yes”, then supporting information on the CHC product will be provided, the consumer record is updated with the approval to purchase CHC product, and the consumer is directed to the distributions choices 317.

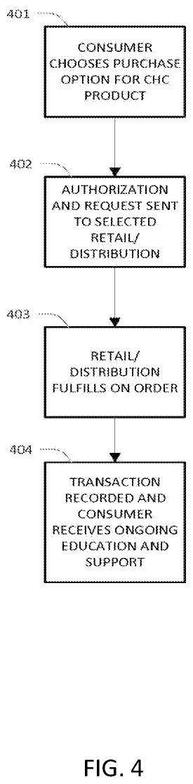

FIG. 4 is one embodiment of a block diagram of one embodiment of the CHC distribution and learning system 104 from FIG. 1. The consumer is given the choice of purchase options 401. These can include but are not limited to the affiliated retail location online or at the retail store. Other delivery and pickup options may be available. Upon choosing the delivery option, an authorization will be sent to the retailer 402. The authorization provides the retailer the approval to fulfill on the order for the consumer 403. Without the authorization, the retailer cannot fulfill the order. The transaction data is collected and can be used for adherence support. The consumer will receive ongoing education and support about CHC product 404. Transaction and consumer information will be recorded in the health record and data repositories as an additive feature of the data set for use in EHRs as well as future studies and public health outcome reports.

Another embodiment of the present invention is in respect to any consumer studies that may be required by the FDA in the development process and before the CHC approval of a drug product. The invention, heretofore described as relating to a drug that has already received CHC approval, will function in much the same way in the simulated CHC settings of a Label Comprehension Study, a Self-Selection Study, an Actual Use Trial, and a Human Factor Study. In each of these studies, a consumer will have access to their personal health record for use in making safe self-selection and usage decisions with respect to the drug product applying for CHC approval.

In summary, the system and method according to an embodiment involves a safety check using product data and consumer data, and a qualification check by consumer Q&A, to make sure that the consumer qualifies to use the OTC, and then an education step, to make sure that the consumer essentially understands all of the label contents that would appear on the drug (uses, dangers, risks, etc.) and, further, a data transfer step to deliver the OTC transaction data to data repositories. This vertically integrated approach is not realized or suggested by any of the existing systems and methods in the OTC products market.

While the present invention has been described at some length and with some particularity with respect to the several described embodiments, it is not intended that it should be limited to any such particulars or embodiments or any particular embodiment, but it is to be construed so as to provide the broadest possible interpretation in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

The functions of the various elements shown in the figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), and non-volatile storage. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams, state transition diagrams, pseudo-code, and the like represent various processes which may be substantially represented in computer readable media and so executed by a computer or processor, whether or not such computer or processor is explicitly shown.

The embodiments of the invention disclosed herein may comprise a system, a method, and/or a computer program product. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device may receive computer readable program instructions from the network and forward the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++, Java, Perl, Python or the like, and conventional procedural programming languages, such as the “C” programming language or similar programming languages. The computer readable program instructions may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and/or computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

A processor or processor circuitry may include a device that has any combination of hardware, circuitry, and software. The hardware and circuitry examples may comprise a parallel processor, a processor array, a vector processor, a scalar processor, a multi-processor, a microprocessor, a communication processor, a network processor, a logic circuit, a queue management device, a central processing unit (CPU), a microprocessing unit (MPU), system on a chip (SoC), a digital signal processor (DSP), an integrated circuit (IC), an application specific integrated circuit (ASIC), a programmable logic device (PLD), and a field programmable gate array (FPGA). A processor or processor circuitry may include one or more processors, one or more circuits and/or software, that responds to and processes basic computer instructions and carries out the instructions of a computer program by performing the basic arithmetic, logical, control and input/output (I/O) operations specified by the instructions, one or more of: an arithmetic logic unit (ALU), which may carry out arithmetic and logic operations on the operands in instructions; a floating point unit (FPU), also known as a math coprocessor or numeric coprocessor, which is a specialized coprocessor that may manipulate numbers more quickly than the basic microprocessor circuitry can in some cases; one or more registers, which may hold instructions and other data and supply operands to the ALU and store the results of operations; and cache memory, which may save time compared to having to get data from random access memory (RAM). A processor or processor circuitry may also include one or more circuits comprising electronic components, such as resistors, memristors, power sources, magnetic devices, motors, generators, solenoids, microphones, speakers, transistors, capacitors, inductors, diodes, semiconductors, switches, antennas, transducers, sensors, detectors, vacuums, tubes, amplifiers, radio receivers, crystals, and oscillators connected by conductive wires or traces through which electric current can flow. The combination of components and wires may allow various simple and complex operations to be performed: signals may be amplified, computations can be performed, and data can be moved from one place to another.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

We claim:

1. A consumer health care product self-selection system, comprising:
a processor coupled to a memory containing program code; and
a network interface configured to provide access to:
a user interface configured to receive input from and providing output to a consumer;
one or more databases;
a product distribution system; and
a product support system;
wherein the processor is configured to:
provide information of one or more products to the consumer via the user interface;
receive selection of the product by the consumer;
determine the consumer's eligibility of purchasing the selected product based on at least one of: consumer data and product data retrieved from the one or more databases, and data entered by the consumer via the user interface;
when the consumer is determined to be eligible, direct the consumer to the product distribution system to purchase the selected product; and
when the consumer is determined to be ineligible, direct the consumer to the product support system to provide product support information of the selected product to the consumer wherein the consumer has direct access to a healthcare professional via but not limited to chat, video conference, telehealth connection or telephone; and
record transaction data to data repositories.

2. The system of claim 1, wherein the information of each of the one or more products comprises:
a) Title (Drug Facts or Drug Facts (continued));
b) Active ingredient(s);
c) Purpose(s);
d) Use(s);
e) Warning(s);
f) Directions;
g) Other information;
h) Inactive ingredients; and, optionally,
i) Questions? or Questions or comments?

3. The system of claim 1, wherein the retrieved data comprises data from at least one of the sources: pharmacy benefit managers (PBMs), health plans, pharmacy data providers, electronic health records (EHR), lab test companies, biometric data sources and other health records.

4. The system of claim 1, wherein the processor is configured to determine the consumer's eligibility by checking for at least one or more of: (i) minimum necessary information on consumer health data being retrieved and matching with consumer-provided identification and purchase rules; (ii) consumer profile predicate matching profile of the product's drug facts label (DFL); (iii) matching of the consumer's health data with the list of drugs and conditions in the Warning(s) section of the product's DFL; (iv) consumer's response to questions about prescriptions that are listed in the consumer's Rx history that are used to treat a certain condition based on the product's DFL; and (v) consumer's response to questions that are not reflected in the consumer's health data.

5. The system of claim 4, wherein the purchase rules comprise limits on at least one of: quantity; time interval; qualifying attempts; number of accounts with same address; and other rules as required to meet FDA requirements.

6. The system of claim 1, wherein the processor is further configured to obtain affirmation from the consumer that the consumer understands the information provided about and instructions for use of the selected product before directing the consumer to the product distribution system to purchase the selected product.

7. The system of claim 1, wherein the processor is further configured to update the consumer data for follow-up education and on-going safe use.

8. The system of claim 1, wherein the eligibility is determined based on at least a set of one or more requirement predicates specific to the labeling of the selected product.

9. The system of claim 8, wherein the requirement predicate is a logical expression that computes a value of “true”, meaning the predicate passes, or “false”, meaning the predicate fails.

10. The system of claim 1, wherein when the consumer is determined to be eligible, the processor is further configured to provide to the consumer information about how to use the selected product, direction and when to stop use.

11. The system of claim 1, wherein when the consumer is determined to be ineligible, the processor is further configured to provide to the consumer a reason of ineligibility and provide access to a healthcare professional via but not limited to chat, video conference, telehealth connection or telephone for further support.

12. A method of providing access of a consumer health care product for safe self-selection and distribution, comprising:
providing information of one or more products to a consumer via a user interface;
receiving selection of a product by the consumer;
determining the consumer's eligibility of purchasing the selected product based on at least one of: consumer data and product data retrieved from one or more databases, and data entered by the consumer via the user interface;
when the consumer is determined to be eligible, directing the consumer to a product distribution system to purchase the selected product; and
when the consumer is determined to be ineligible, directing the consumer to a product support system to provide product support information of the selected product to the consumer; provide access to a healthcare professional;
recording transaction data to data repositories.

13. The method of claim 12, wherein the information of each of the one or more products comprises:
a) Title (Drug Facts or Drug Facts (continued));
b) Active ingredient(s);
c) Purpose(s);
d) Use(s);
e) Warning(s);
f) Directions;
g) Other information;
h) Inactive ingredients; and, optionally,
i) Questions? or Questions or comments?

14. The method of claim 12, wherein the retrieved data comprises data from at least one of the sources: pharmacy benefit managers (PBMs), health plans, pharmacy data providers, electronic health records (EHR), lab test companies, biometric data sources and other health records.

15. The method of claim 12, further comprising determining the consumer's eligibility by checking for at least one or more of: (i) minimum necessary information on consumer health data being retrieved and matching with consumer-provided identification and purchase rules; (ii) consumer profile predicate matching profile of the product's drug facts label (DFL); (iii) matching of the consumer's health data with the list of drugs and conditions in the Warning(s) section of the product's DFL; (iv) consumer's response to questions about prescriptions that are listed in the consumer's Rx history that are used to treat a certain condition based on the product's DFL; and (v) consumer's response to questions that are not reflected in the consumer's health data.

16. The system of claim 15, wherein the purchase rules comprise limits on at least one of: quantity; time interval; qualifying attempts; number of accounts with same address; and other rules as required to meet FDA requirements.

17. The method of claim 12, further comprising obtaining affirmation from the consumer that the consumer understands the information provided about and instructions for use of the selected product before directing the consumer to the product distribution system to purchase the selected product.

18. The method of claim 12, further comprising updating the consumer data for follow-up education and on-going safe use.

19. The method of claim 12, wherein the determining the consumer's eligibility is based on at least a set of one or more requirement predicates specific to the labeling of the selected product.

20. The method of claim 19, wherein the requirement predicate is a logical expression that computes a value of “true”, meaning the predicate passes, or “false”, meaning the predicate fails.
